(19) United States
(12) Patent Application Publication
(10) Pub. No.: US 2020/0003748 A1
(43) Pub. Date: Jan. 2, 2020

(54) DETECTION DEVICE FOR ENVIRONMENTALLY HARMFUL PARTICLES

(71) Applicant: Siqi YI, Shuanglong Village (CN)

(72) Inventor: Siqi YI, Shuanglong Village (CN)

(21) Appl. No.: 16/021,501

(22) Filed: Jun. 28, 2018

(51) Int. Cl. G01N 33/24; G01N 33/00

(52) U.S. Cl. CPC G01N 33/24; G01N 33/0027

(57) ABSTRACT

The present application discloses a detection device for environmentally harmful particles, including: a detection device body, an electric universal wheel, a battery, a cavity, a first electric telescopic rod, an air quality sensor, an electric push rod, a push plate, a base plate, a slide rail, a pulley, a hinge, a storage box, an electric jack, a pad, an outlet, a PLC controller, a second electric telescopic rod, a solid-liquid sensor and an excavator. The present application has a scientific and reasonable structure and is safe and convenient to use; the first electric telescopic rod and the air quality sensor are provided to facilitate the detection of harmful particles in the air by the detection device; the excavator is provided to facilitate the collection of solid-liquid mixture to be detected; the storage box is provided to facilitate the collection and storage of the solid-liquid mixture to be detected, so as to facilitate the next step; the second electric telescopic rod and the solid-liquid sensor are provided to facilitate the detection of the solid-liquid mixture to be detected in the storage box; members, such as the electric push rod and the electric jack, are provided to facilitate the movement and cleaning of the storage box.

### TECHNICAL FIELD

The present application relates to the technical field of detection device, in particular to a detection device for environmentally harmful particles.

### BACKGROUND

With the rapid development of economy and society, environmental issues have become increasingly prominent. Environmental issues such as PM10, PM2.5, TSP, greenhouse effect, and energy conservation and emission reduction have become a focused issue of countries and people around the world. More and more ordinary people desiderate a household detector to monitor daily life water and atmospheric environment, as to ensure the quality of the living environment.

Current household detection devices for harmful particles have the following disadvantages, such as long detection time, inconvenience for use, complicated structure, high cost, inconvenience for promotion to residents, and unsafe use.

### SUMMARY

The present application provides a detection device for environmentally harmful particles which can effectively overcome the disadvantages of the above background.

To achieve the above objectives, the present application provides the following technical solution: a detection device for environmentally harmful particles, including: a detection device body, an electric universal wheel, a battery, a cavity, a first electric telescopic rod, an air quality sensor, an electric push rod, a push plate, a base plate, a slide rail, a pulley, a hinge, a storage box, an electric jack, a pad, an outlet, a PLC controller, a second electric telescopic rod, a solid-liquid sensor and an excavator, wherein the electric universal wheel is fixed on a bottom of the detection device body; the battery is fixed on one side of the bottom of the detection device body; the cavity is provided on one side of a top of the detection device body, above the battery; the first electric telescopic rod is provided at an inner side of a bottom of the cavity; the air quality sensor is installed at a top of the first electric telescopic rod; the electric push rod is installed at one end of the battery; the push plate is fixed at one end of the electric push rod; the base plate is fixed at a bottom of the push plate; the slide rail is provided below the base plate; a bottom of the base plate is installed with the pulley on an inner side of the slide rail; the hinge is fixed on one side of a top of the base plate; the storage box is connected to the one side of the top of the base plate via the hinge; the electric jack is connected between one side of a bottom of the storage box and other side of the top of the base plate; the pad is fixed on an outer side of the top of the base plate; the outlet is provided in a middle of other side of the detection device body; the PLC controller is medially embedded in an outer side of the top of the detection device body; the second electric telescopic rod is medially installed on an inner side of the top of the detection device body, right above the storage box; the solid-liquid sensor is installed on a bottom of the second electric telescopic rod; the excavator is installed on other side of the top of the detection device body; the detection device body, the electric universal wheel, the first electric telescopic rod, the air quality sensor, the electric push rod, the electric jack, the PLC controller, the second electric telescopic rod, the solid-liquid sensor and the excavator are all electrically connected with the battery; output ends of the air quality sensor and the solid-liquid sensor are electrically connected with an input end of the PLC controller; input ends of the electric universal wheel, the first electric telescopic rod, the electric push rod, the electric jack, the second electric telescopic rod and the excavator are all electrically connected with an output end of the PLC controller.

Compared to prior arts, the present application has the following advantages: the present application has a scientific and reasonable structure and is safe and convenient to use; the first electric telescopic rod and the air quality sensor are provided to facilitate the detection of harmful particles in the air by the detection device; the excavator is provided to facilitate the collection of solid-liquid mixture to be detected; the storage box is provided to facilitate the collection and storage of the solid-liquid mixture to be detected, so as to facilitate the next step; the second electric telescopic rod and the solid-liquid sensor are provided to facilitate the detection of the solid-liquid mixture to be detected in the storage box; members, such as the electric push rod and the electric jack, are provided to facilitate the movement and cleaning of the storage box.

### BRIEF DESCRIPTION OF THE DRAWINGS

FIG. 1 is a schematic structural view of the present application;

FIG. 2 is a schematic structural view of section A in FIG. 1 of the present application; and

FIG. 3 is a schematic structural view of the excavator of the present application.

### DETAILED DESCRIPTION OF THE EMBODIMENTS

The preferred embodiments of the present application are described below with reference to the accompanying drawings. It should be appreciated that the preferred embodiments described herein are only illustrative and explanatory of the present application and are not intended to limit the present application.

### Embodiment

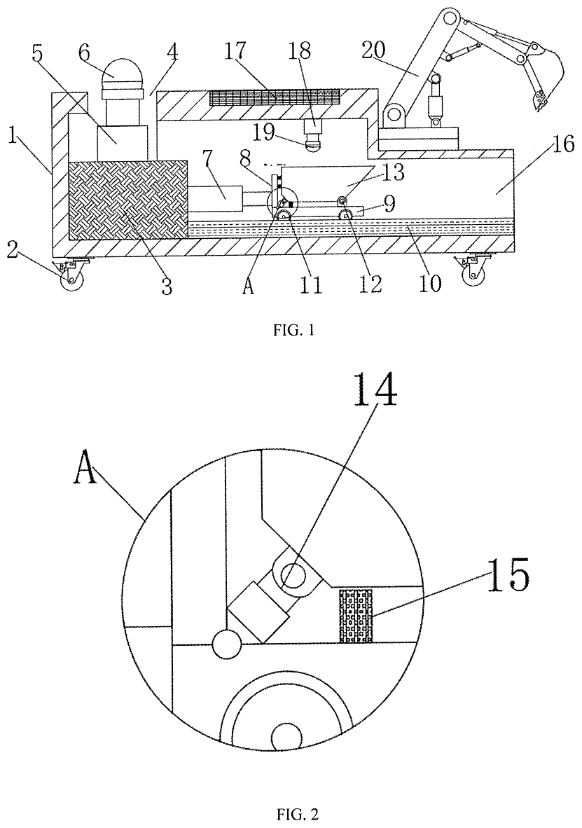

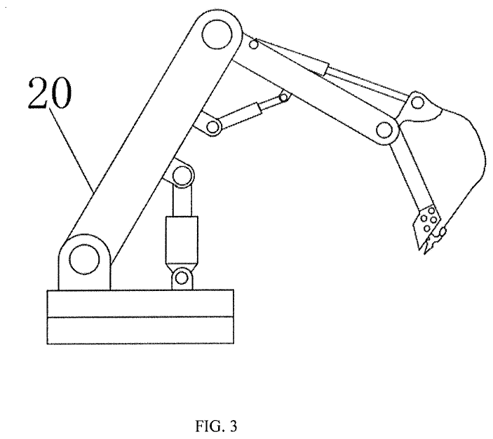

As shown in FIGS. 1-3, the present application discloses a detection device for environmentally harmful particles, including: a detection device body 1, an electric universal wheel 2, a battery 3, a cavity 4, a first electric telescopic rod 5, an air quality sensor 6, an electric push rod 7, a push plate 8, a base plate 9, a slide rail 10, a pulley 11, a hinge 12, a storage box 13, an electric jack 14, a pad 15, an outlet 16, a PLC controller 17, a second electric telescopic rod 18, a solid-liquid sensor 19 and an excavator 20. The electric universal wheel 2 is fixed on the bottom of the detection device body 1; the battery 3 is fixed on one side of the bottom of the detection device body 1; the cavity 4 is provided on one side of the top of the detection device body 1, above the battery 3; the first electric telescopic rod 5 is provided at an inner side of the bottom of the cavity 4; the air quality sensor 6 is installed at the top of the first electric telescopic rod 5; the electric push rod 7 is installed at one end of the battery 3; the push plate 8 is fixed at one end of the electric push rod 7; the base plate 9 is fixed at a bottom of the push plate 8; the slide rail 10 is provided below the base plate 9; a bottom of the base plate 9 is installed with the pulley 11 on the inner side of the slide rail 10; the hinge 12 is fixed on one side of the top of the base plate 9; the storage box 13 is connected to the one side of the top of the base plate 9 via the hinge 12; the electric jack 14 is connected between one side of the bottom of the storage box 13 and the other side of the top of the base plate 9; the pad 15 is fixed on the outer side of the top of the base plate 9; the outlet 16 is provided in the middle of the other side of the detection device body 1; the PLC controller 17 is medially embedded in the outer side of the top of the detection device body 1; the second electric telescopic rod 18 is medially installed on the inner side of the top of the detection device body 1, right above the storage box 13; the solid-liquid sensor 19 is installed on the bottom of the second electric telescopic rod 18; the excavator 20 is installed on the other side of the top of the detection device body 1. The detection device body 1, the electric universal wheel 2, the first electric telescopic rod 5, the air quality sensor 6, the electric push rod 7, the electric jack 14, the PLC controller 17, the second electric telescopic rod 18, the solid-liquid sensor 19 and the excavator 20 are all electrically connected with the battery 3. Output ends of the air quality sensor 6 and the solid-liquid sensor 19 are electrically connected with an input end of the PLC controller 17. Input ends of the electric universal wheel 2, the first electric telescopic rod 5, the electric push rod 7, the electric jack 14, the second electric telescopic rod 18 and the excavator 20 are all electrically connected with an output end of the PLC controller 17.

In order to facilitate the protection of the battery 3, in the present embodiment, preferably, a protective cover is installed outside the battery 3.

In order to make the first electric telescopic rod 5 work better, in the present embodiment, preferably, the bottom end of the first electric telescopic rod 5 is medially installed on the top of the battery 3.

In order to increase the elasticity of the pad 15, in the present embodiment, preferably, the pad 15 is a member of rubber.

In order to facilitate the reduction of the friction at a joint between the slide rail 10 and the pulley 11 and the hinge 12, in the present embodiment, preferably, both the joint between the slide 10 and the pulley 11 and the interior of the hinge 12 are provided with lubricating oil.

The working principle and use process of the present application are as follows: when detecting air, the PLC controller 17 controls the electric universal wheel 2 to move such that the detection device body 1 moves to the position to be detected. Then the first electric telescopic rod 5 is extended and retracted by the PLC controller 17, and the extension and retraction of the first electric telescopic rod 5 drives the air quality sensor 6 to rise from the cavity 4. When the air quality sensor 6 rises to a predetermined height, the first electric telescopic rod 5 stops extending and retracting, then the air quality sensor 6 detects and transmits the detection signal to the PLC controller 17; when detecting solid-liquid mixture, the PLC controller 17 controls the electric universal wheel 2 to move such that the detection device body 1 moves to the position to be detected. Then the electric push rod 7 is controlled to push the push plate 8 to move toward the outlet 16, and the movement of the push plate 8 drives the base plate 9 to move on the slide rail 10 via the pulley 11, and the movement of the push plate 8 drives the storage box 13 to move toward the outlet 16. When the storage box 13 is moved to the appropriate position at the outlet 16, the electric push rod 7 stops pushing, thereafter the excavator 20 is initiated to deliver the solid particles or liquid to be detected to the storage box 13 through the excavator 20. The electric push rod 7 is pulled back, and the pulling back of the electric push rod 7 drives the base plate 9 and the storage box 13 to move back via the push plate 8. When the storage box 13 reaches the original position, the electric push rod 7 stops pulling back. Finally, the second electric telescopic rod 18 is further extended and retracted to move the solid-liquid sensor 19 toward the storage box 13. When the solid-liquid sensor 19 arrives at an appropriate position inside the storage box 13, the second electric telescopic rod 18 stops extending and retracting, and the solid-liquid sensor 19 detects and transmits the detection signal to the PLC controller 17. The electric jack 14 is provided to facilitate the cleaning of the internal waste of the storage box 13. This design has the following advantages, such as short detection time, convenience for use, simple structure, reasonable cost, convenience for promotion to residents and safe use.

The above description is only the preferred embodiments of the present application and is not intended to limit the present application. Any modification, equivalent replacement and improvement made within the spirit and principle of the present application should be included in the scope of the present application.

What is claimed is:

1. A detection device for environmentally harmful particles, comprising: a detection device body, an electric universal wheel, a battery, a cavity, a first electric telescopic rod, an air quality sensor, an electric push rod, a push plate, a base plate, a slide rail, a pulley, a hinge, a storage box, an electric jack, a pad, an outlet, a PLC controller, a second electric telescopic rod, a solid-liquid sensor and an excavator, wherein the electric universal wheel is fixed on a bottom of the detection device body; the battery is fixed on one side of the bottom of the detection device body; the cavity is provided on one side of a top of the detection device body, above the battery; the first electric telescopic rod is provided at an inner side of a bottom of the cavity; the air quality sensor is installed at a top of the first electric telescopic rod; the electric push rod is installed at one end of the battery; the push plate is fixed at one end of the electric push rod; the base plate is fixed at a bottom of the push plate; the slide rail is provided below the base plate; a bottom of the base plate is installed with the pulley on an inner side of the slide rail; the hinge is fixed on one side of a top of the base plate; the storage box is connected to the one side of the top of the base plate via the hinge; the electric jack is connected between one side of a bottom of the storage box and other side of the top of the base plate; the pad is fixed on an outer side of the top of the base plate; the outlet is provided in a middle of other side of the detection device body; the PLC controller is medially embedded in an outer side of the top of the detection device body; the second electric telescopic rod is medially installed on an inner side of the top of the detection device body, right above the storage box; the solid-liquid sensor is installed on a bottom of the second electric telescopic rod; the excavator is installed on other side of the top of the detection device body; the detection device body, the electric universal wheel, the first electric telescopic rod, the air quality sensor, the electric push rod, the electric jack, the PLC controller, the second electric telescopic rod, the solid-liquid sensor and the excavator are all electrically connected with the battery; output ends of the air quality sensor and the solid-liquid sensor are electrically connected with an input end of the PLC controller;
input ends of the electric universal wheel, the first electric telescopic rod, the electric push rod, the electric jack, the second electric telescopic rod and the excavator are all electrically connected with an output end of the PLC controller.
